(19) United States
(12) Patent Application Publication
(10) Pub. No.: US 2012/0323199 A1
(43) Pub. Date: Dec. 20, 2012

(54) Leakage Proof Base Material for Paper Diaper

(76) Inventors: Po-An Tsai, Kaohsiung City (TW); Charng-Ching Ou, Taipei City (TW); Hsu-Feng Shih, Changhua County (TW)

(21) Appl. No.: 13/369,286

(22) Filed: Feb. 8, 2012

Related U.S. Application Data
(63) Continuation-in-part of application No. 13/163,778, filed on Jun. 20, 2011.

(30) Foreign Application Priority Data
Aug. 16, 2011 (TW) 100215492

(51) Int. Cl. A61L 15/28; A61L 15/26; A61L 15/24

(52) U.S. Cl. 604/372; 604/375

(57) ABSTRACT

A leakage proof base material for paper diaper is provided which is more convenient to use, suitable to be mass produced, more skin-friendly and with a lower cost. The leakage proof base material for paper diaper comprises a leakage proof base material which is composed of a leakage proof membrane as a top layer and a non-textile fabric layer as a bottom layer, and surfaces of the leakage proof membrane and the non-textile fabric layer are combined together partially or entirely. At least one through hole area is disposed on the non-textile fabric layer, the through hole area is composed of a plurality of through holes which are penetrated through the non-textile fabric layer and are spaced at intervals.

### CROSS-REFERENCE TO RELATED APPLICATIONS

In accordance to the privileges granted under 35 U.S.C. §119(b), this United States nonprovisional application claims the priority benefit of the Taiwanese patent application serial number 100215492 filed on Aug. 16, 2011. Disclosure of the cross-referenced application identified above is incorporated by reference in its entirety. In addition, this nonprovisional application is a continuation-in-part of the U.S. nonprovisional application Ser. No. 13/163,778, filed on Jun. 20, 2011 entitled “DIAPER WITH IMPROVED CONJUGATION STRUCTURE.” Disclosure of the application identified above is also incorporated by reference in its entirety.

### BACKGROUND OF THE INVENTION

1. Field of Invention

The present invention relates to a leakage proof base material for paper diaper.

2. Related Art

Cloth diaper is commonly used for wrapping around the bottoms of a baby or for laying on a baby bed for receiving the urine of the baby. The cloth diaper is like a second layer of skin for the baby from the beginning until the baby is grown up to be able to go to the toilet by himself or herself.

Diaper can be divided into two types in terms of material, which are the cloth diaper and the paper diaper. The advantages of the cloth diaper are: it has a lower price, can be used repeatedly, has better absorbance and tightness, the baby skin is less allergic to it and less likely to be affected with diaper rash; the drawbacks of the cloth diaper are: inconvenient to wash and to be carried around. The advantages of the paper diaper are: convenient to be carried around and disposable; the drawbacks of the paper diaper are: it has to be changed frequently for babies with sensitive skin and can only be used once.

As society and economic are developing rapidly, the ways and tools for raising a baby have changed accordingly and disposable paper diaper is more commonly used. Besides that the paper diaper has many advantages in terms of its convenience, functions and practicality, the development of mental health and personality of the baby can also be benefited.

Many types of paper diaper are found in the market today as technology is developing and the standard of living is enhanced. In order to avoid the stickiness between the conventional paper diaper and human skin, a type of paper diaper with a leakage proof base material composed of a top plastic membrane (A) and a bottom non-textile fabric layer (B1) is introduced into the market as shown in FIG. 1, so that the paper diaper can be more skin-friendly and the discomfort of skin of the users can be reduced.

The applicant of the present invention has invented a paper diaper with an improved combining structure in the Taiwan patent publication number M405257. A leakage proof base material composed of a top plastic membrane (A) and a bottom non-textile fabric layer (B2) with a rough surface are applied in the paper diaper with an improved combining structure as shown in FIG. 2, by which the way of fixing the paper diaper on the waist of the user can be improved, the cost can be reduced, the amount of material used can be saved, the defect-free rate and the skin-friendliness can be enhanced.

Even though the leakage proof base material employed in the invention with Taiwan patent publication number M405257 is more skin-friendly, the fixing method is innovative and the cost is also reduced, the leakage proof base material is not in the best disposition. Furthermore, improvements can still be made in terms of the cost, the amount of material used, the skin-friendliness and the way of fixing the paper diaper.

In view of the abovementioned, a leakage proof base material for paper diaper of the present invention is provided to achieve the advantages of reducing the cost and the amount of material used, enhancing the skin-friendliness and improving the way of fixing the paper diaper.

### SUMMARY

An embodiment of the present invention is to provide a leakage proof base material for paper diaper which is more convenient to use, suitable to be mass produced, more skin-friendly and with a lower cost.

In order to solve abovementioned problems and achieve the abovementioned benefits, the technical means of the present invention includes providing a leakage proof base material for paper diaper which comprises: a leakage proof base material composed of a leakage proof membrane as a top layer and a non-textile fabric layer as a bottom layer, and surfaces of the leakage proof membrane and the non-textile fabric layer are combined together partially or entirely; at least one through hole area is disposed on the non-textile fabric layer, the through hole area is composed of a plurality of through holes which are penetrated through the non-textile fabric layer and are spaced at intervals.

According to the leakage proof base material for paper diaper, the leakage proof membrane and the non-textile fabric layer are either an elastic layer or a non-elastic layer or a combination of them; and the elasticity coefficients of the leakage proof membrane and the non-textile fabric layer can be different.

According to the leakage proof base material for paper diaper, the leakage proof membrane is either an air permeable leakage proof membrane or an air impermeable leakage proof membrane based on the degree of air permeability.

According to the leakage proof base material for paper diaper, the leakage proof membrane is either a polypropylene (PP) leakage proof membrane, a polyethylene (PE) leakage proof membrane, a polyurethane (PU) leakage proof membrane or a plastic composite leakage proof membrane of decomposable plastic and copolymer.

According to the leakage proof base material for paper diaper, the through hole areas are disposed on the non-textile fabric layer in one of the ways of: equally spaced at intervals, unequally spaced at intervals, covering the surface entirely, covering the surface partially, covering the central surface of the non-textile fabric layer entirely, covering the central surface of the non-textile fabric layer partially, covering the central surface of the non-textile fabric layer in equally spaced intervals and covering the central surface of the non-textile fabric layer in unequally spaced intervals.

According to the leakage proof base material for paper diaper, the through hole area is disposed in one of the shapes of: circular area, character area, strip area, rectangular area, pattern area, dot area, tartan area, regular striation area and irregular striation area.

Comparing with the effects of the conventional techniques:

1) The surface of the leakage proof base material for paper diaper of the present invention is more rough and uneven by the disposition of the through hole area on the non-textile fabric layer. Because the area in contact with the skin is reduced, the volume of the three dimensional space between the material and the skin is increased, so that air can be circulated more freely and the discomfort can be reduced when the skin is in contact with the leakage proof base material of the present invention. The comfort provided by the present invention is like that of the cloth diaper made by textile fabric or cotton. Therefore, better skin-friendliness can be achieved and the stickiness between the conventional plastic diaper and the skin can be eliminated.

2) In the present invention, the through hole area is formed by disposing a plurality of the through holes on the non-textile fabric layer. The production cost of the leakage proof base material can be reduced and the production can be optimized because the material for conventional loops can be saved with the through holes design. As a result, the overall cost can be reduced, materials and energy consumption can be saved and the emission of carbon can be reduced.

3) The surface of the leakage proof base material for paper diaper of the present invention is even more rough and uneven by the disposition of the through holes on the non-textile fabric layer, and the non-textile fabric layer can have a three dimensional nature to be cooperated with hooks for fixing the paper diaper on the waist. Therefore, besides that the conventional loops for fixing are not required by the present invention, an adhering effect better than that of the invention with Taiwan patent publication number M405257 can be achieved, and can be cooperated with the hooks for providing a better fixing effect for fixing the paper diaper on the waist.

The present invention will become more fully understood by reference to the following detailed description thereof when read in conjunction with the attached drawings.

### BRIEF DESCRIPTION OF THE DRAWINGS

FIG. 1 is a cross-sectional view of a conventional leakage proof base material.

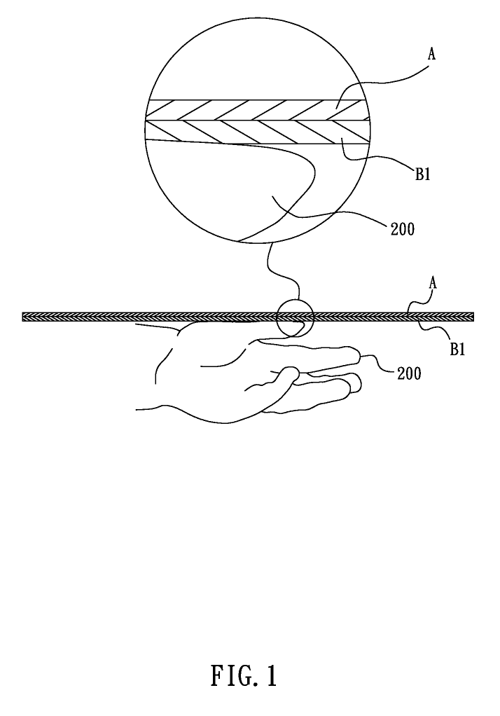

FIG. 2 is a cross-sectional view of a leakage proof base material.

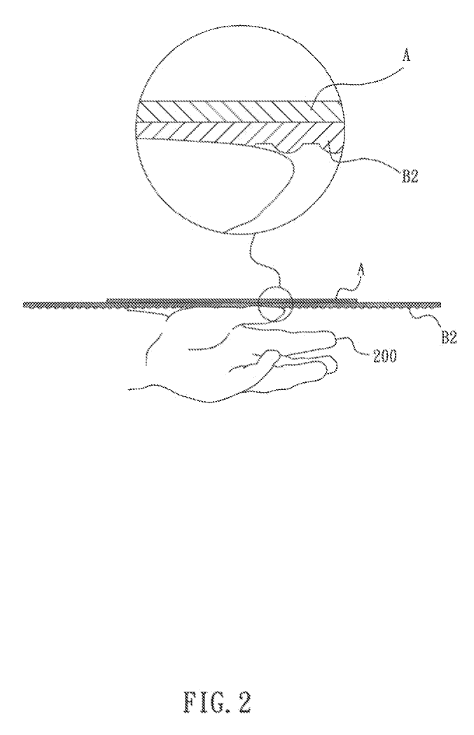

FIG. 3 is a perspective view of a first model of a leakage proof base material for paper diaper of the present invention.

FIG. 4 is an exploded perspective view of the first model of the leakage proof base material for paper diaper of the present invention.

FIG. 5 is a cross-sectional view of the first model of the leakage proof base material for paper diaper of the present invention.

FIG. 6 is a front view of the first model of the leakage proof base material for paper diaper of the present invention.

FIG. 7 is a cross-sectional partial enlarged view of FIG. 6.

FIG. 8 is a front view of a second model of the leakage proof base material for paper diaper of the present invention.

FIG. 9 is a cross-sectional partial enlarged view of FIG. 8.

FIG. 10 is a front view of a third model of the leakage proof base material for paper diaper of the present invention.

FIG. 11 is a cross-sectional partial enlarged view of FIG. 10.

FIG. 12 is a front view of a fourth model of the leakage proof base material for paper diaper of the present invention.

FIG. 13 is a cross-sectional partial enlarged view of FIG. 12.

FIG. 14 is a front view of a fifth model of the leakage proof base material for paper diaper of the present invention.

FIG. 15 is a cross-sectional partial enlarged view of FIG. 14.

FIGS. 16 and 17 are perspective views of the first model of the leakage proof base material for paper diaper of the present invention being embodied on a paper diaper.

FIGS. 18 and 19 are perspective views of the second model of the leakage proof base material for paper diaper of the present invention being embodied on a paper diaper.

FIGS. 20 and 21 are perspective views of the third model of the leakage proof base material for paper diaper of the present invention being embodied on a paper diaper.

FIGS. 22 and 23 are perspective views of the fourth model of the leakage proof base material for paper diaper of the present invention being embodied on a paper diaper.

FIGS. 24 and 25 are perspective views of the fifth model of the leakage proof base material for paper diaper of the present invention being embodied on a paper diaper.

Corresponding reference characters indicate corresponding parts throughout the drawings.

### DETAILED DESCRIPTION

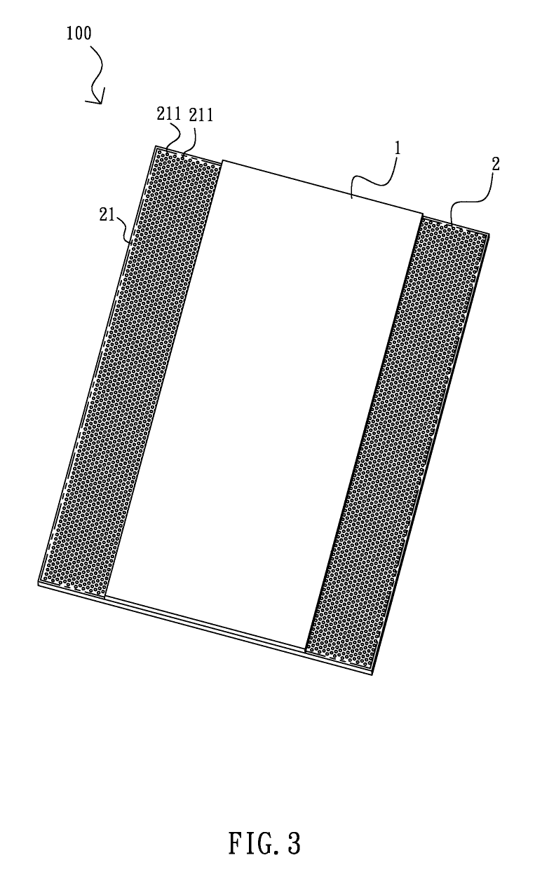

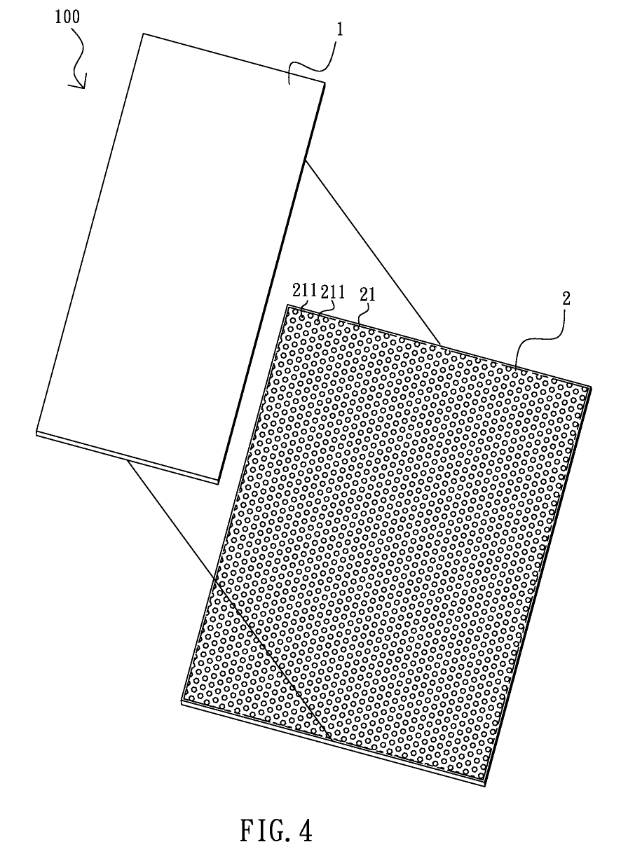

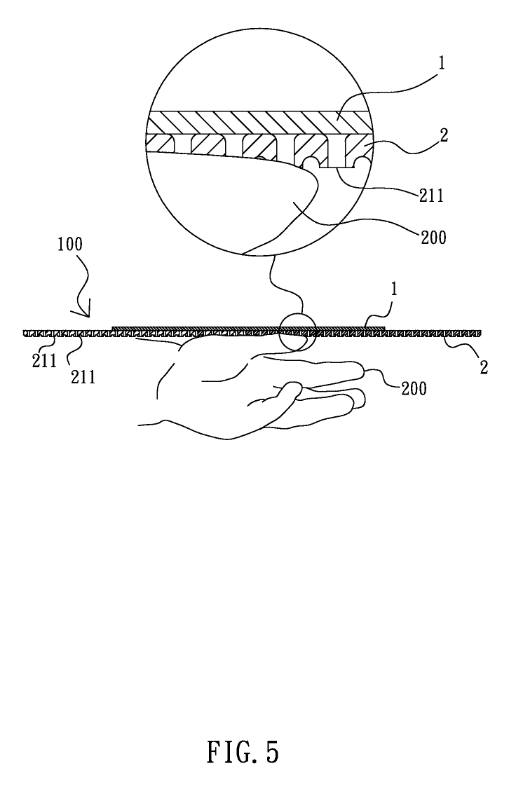

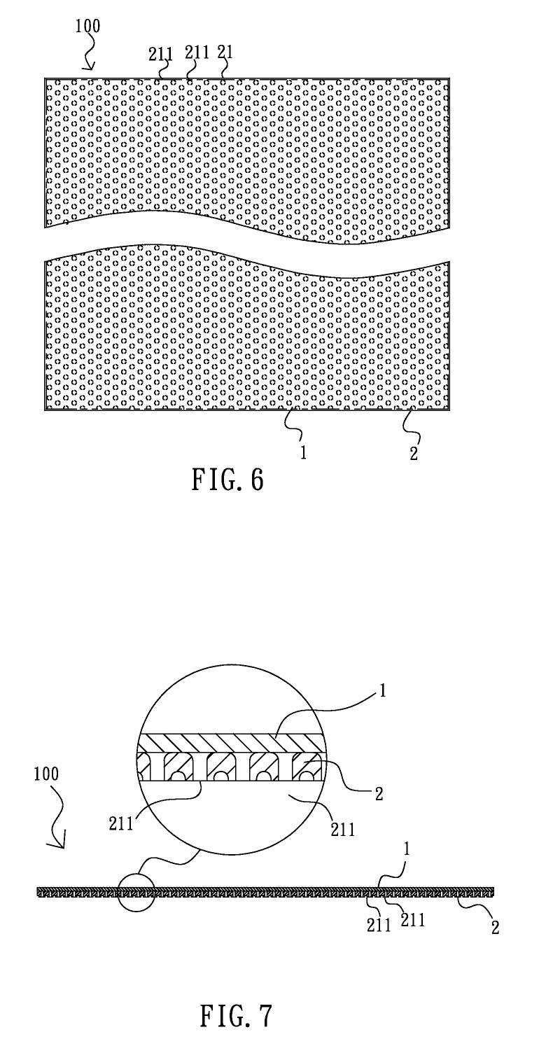

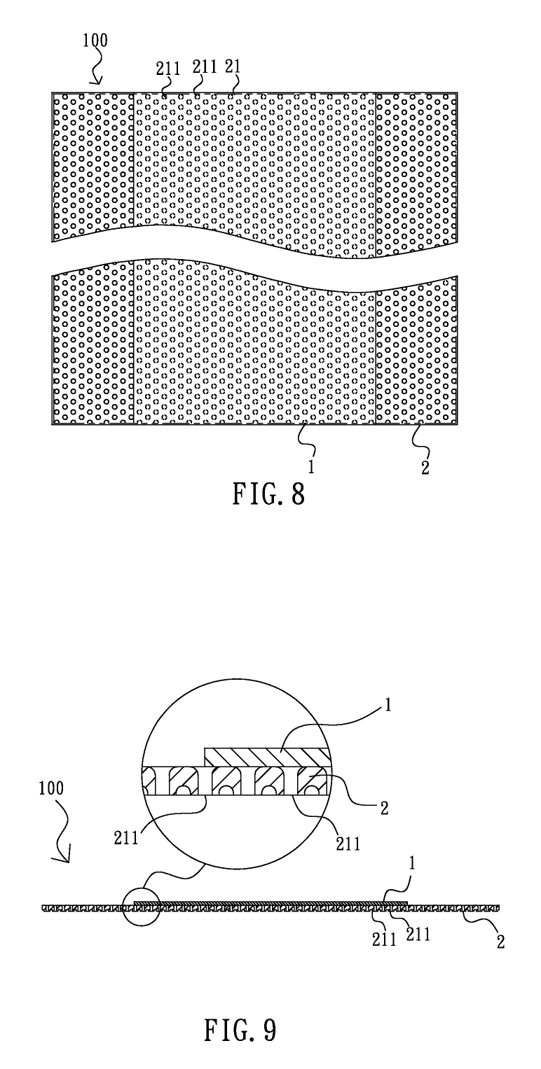

FIG. 3 is a perspective view of a first model of a leakage proof base material for paper diaper of the present invention; FIG. 4 is an exploded perspective view of the first model of the leakage proof base material for paper diaper of the present invention; FIG. 5 is a cross-sectional view of the first model of the leakage proof base material for paper diaper of the present invention; FIG. 6 is a front view of the first model of the leakage proof base material for paper diaper of the present invention; and FIG. 7 is a cross-sectional partial enlarged view of FIG. 6.

As shown in the drawings, the leakage proof base material for paper diaper of the present invention includes a leakage proof base material 100 composed of a leakage proof membrane 1 as a top layer and a non-textile fabric layer 2 as a bottom layer, and surfaces of the leakage proof membrane 1 and the non-textile fabric layer 2 are combined together partially or entirely. At least one through hole area 21 is disposed on the non-textile fabric layer 2, the through hole area 21 is composed of a plurality of through holes 211 which are penetrated through the non-textile fabric layer 2 and are spaced at intervals.

When the present invention is applied in a paper diaper and the paper diaper is worn on a baby, an outer layer of the paper diaper is the non-textile fabric layer 2. The non-textile fabric layer 2 is presented in a three dimensional nature with an uneven surface of convex and concave structures because of the disposition of the through holes 211 on the through hole area 21. Because of the contact area between the skin and the non-textile fabric layer 2 is reduced, the volume of the three dimensional space between the non-textile fabric layer 2 and the skin is increased, so that air can be circulated more freely and the texture provided by the present invention is like that of the textile fabric. Therefore, the baby or an adult who wear the paper diaper can feel more comfortable when the skin is in contact with the non-textile fabric layer 2. As a result, the overall skin-friendliness can be enhanced, and the stickiness between the conventional plastic material and the skin can be eliminated.

With the disposition of the through holes 211 of the through hole area 21, besides that the skin can feel more comfortable, the holding power between the non-textile fabric layer 2 and the leakage proof membrane 1 can be enhanced. Furthermore, besides that the look is more aesthetically pleasing, the contact area between the non-textile fabric layer 2 and hooks can be increased in order to have the hooking or adhering effect enhanced because the non-textile fabric layer 2 with the through holes 211 is structurally similar to that of conventional loops. As a result, the non-textile fabric layer 2 can be hooked or adhered with the hooks repeatedly without having to use the loops.

Furthermore, the through holes 211 on the non-textile fabric layer 2 are formed by punching with needles, and a number of the through holes 211 and the form of the through holes 211 being presented can be achieved by adjusting the disposition of the needles. Therefore, the material with the conventional loops is not required anymore for mass production of the paper diapers, and the material is not required to be produced by the machines. As a result, the production efficiency is enhanced, the cost of materials and the defective percentage can be reduced, the overall costs and the amount of materials can be reduced and the emission of carbon can be reduced as well.

The bladder of new born babies has not grown to full size yet, therefore urine can not be hold for a long time and the paper diaper has to be changed many times from once every half an hour to four times daily averagely. The paper diaper has to be changed six to seven times daily and gradually to four times daily as the baby grow up, therefore the amount of paper diapers used daily is large. Besides that the present invention is convenient to use, the financial burden on the users can be relieved by employing paper diapers with the leakage proof base material 100 of the present invention because of its lower production cost.

The leakage proof membrane 1 and the non-textile fabric layer 2 are either an elastic layer or a non-elastic layer or a combination of them; and the elasticity coefficients of the leakage proof membrane 1 and the non-textile fabric layer 2 can be different.

By employing the leakage proof membrane 1 and the non-textile fabric layer 2, the discomfort of the baby can be reduced, the paper diaper is more fitted on the baby and the baby can be moved freely.

By employing the leakage proof membrane 1 and the non-textile fabric layer 2, besides that the discomfort of the baby can be reduced and the paper diaper is more fitted on the baby, the paper diaper is prevented to be displaced or detached when the baby or adult is in motion, and the degree of irritation on the skin of the users can be reduced tremendously.

The leakage proof membrane 1 is either an air permeable leakage proof membrane or an air impermeable leakage proof membrane based on the degree of air permeability.

The type of the leakage proof membrane 1 being employed is based on the characteristics of the paper diaper. If the air permeable leakage proof membrane is used as the leakage proof membrane 1, the air permeability of the present invention can be enhanced which is suitable for used in summer to reduce the possibility of the skin of baby and adult being infected with diaper rash and allergy.

If the air impermeable leakage proof membrane is used as the leakage proof membrane 1, the possibility of urine leakage can be reduced and also the degree of waterproof can be enhanced.

The leakage proof membrane 1 is either a polypropylene (PP) leakage proof membrane, a polyethylene (PE) leakage proof membrane, a polyurethane (PU) leakage proof membrane or a plastic composite leakage proof membrane of decomposable plastic and copolymer.

The type of the leakage proof membrane 1 being employed is based on the characteristics of the paper diaper. The material of the leakage proof membrane 1 being employed is based on the requirements of application of the paper diaper.

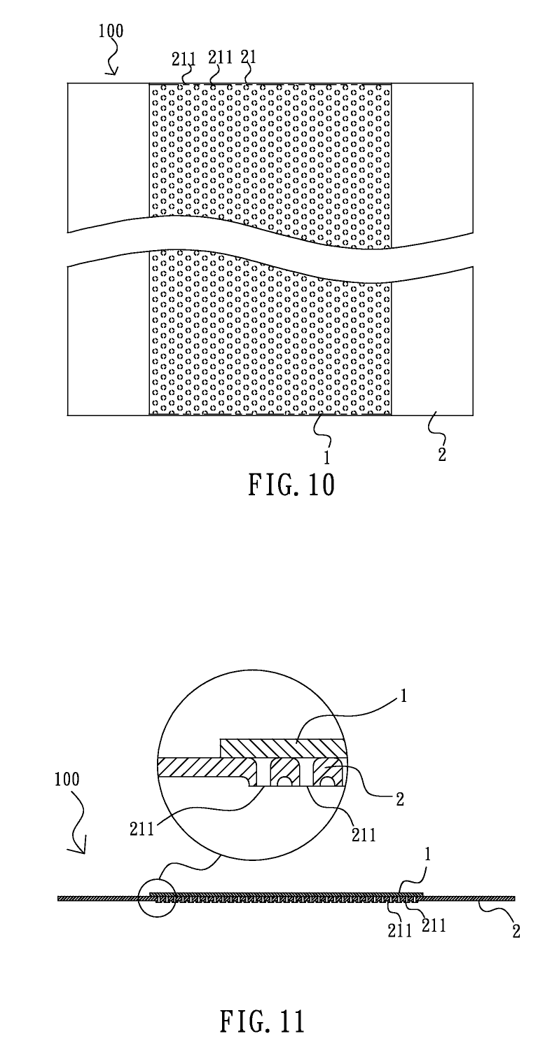

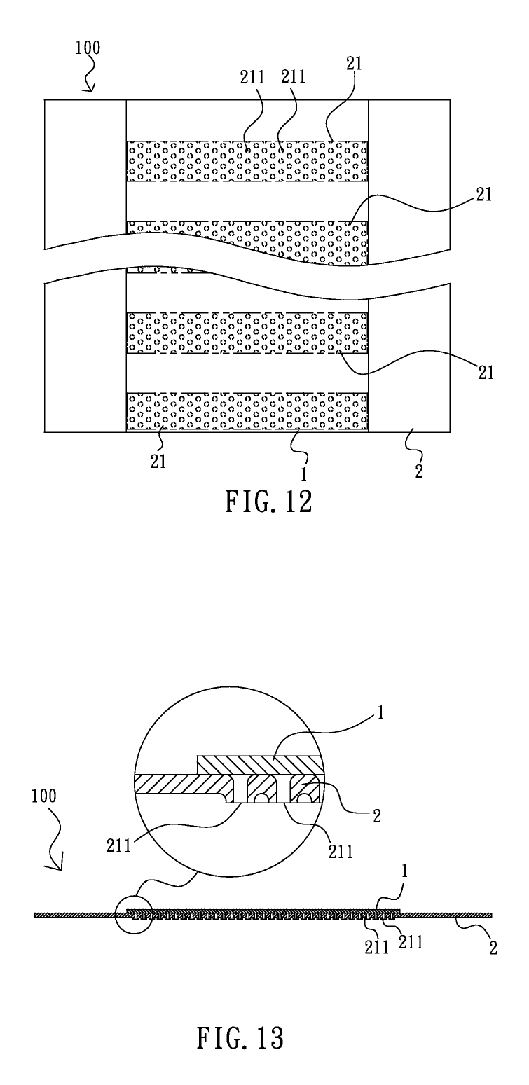

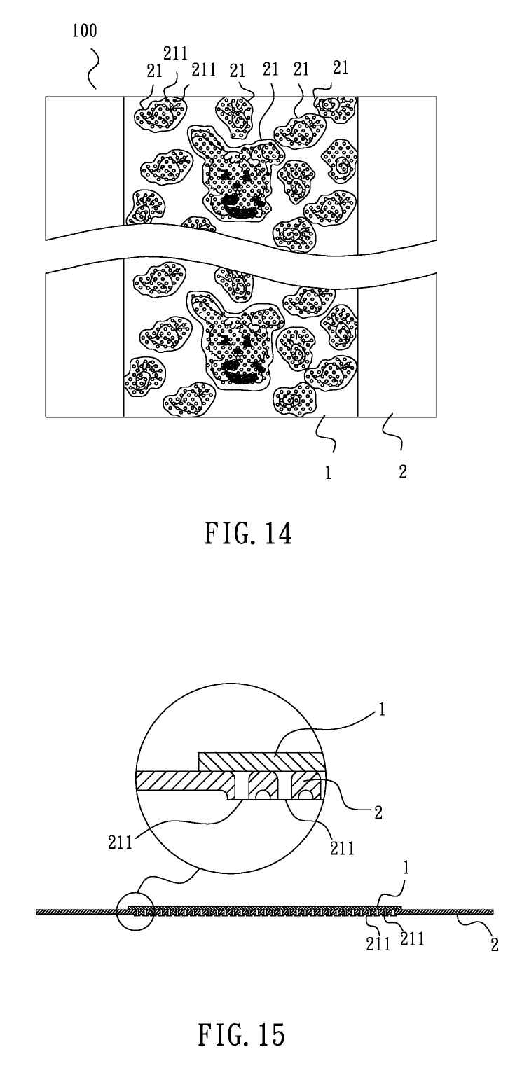

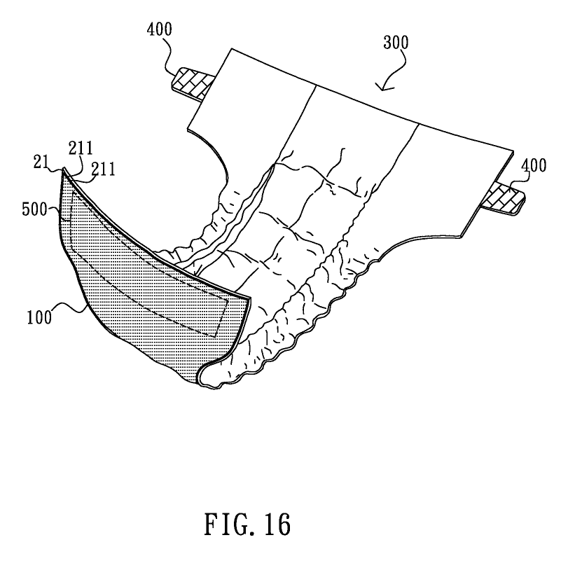

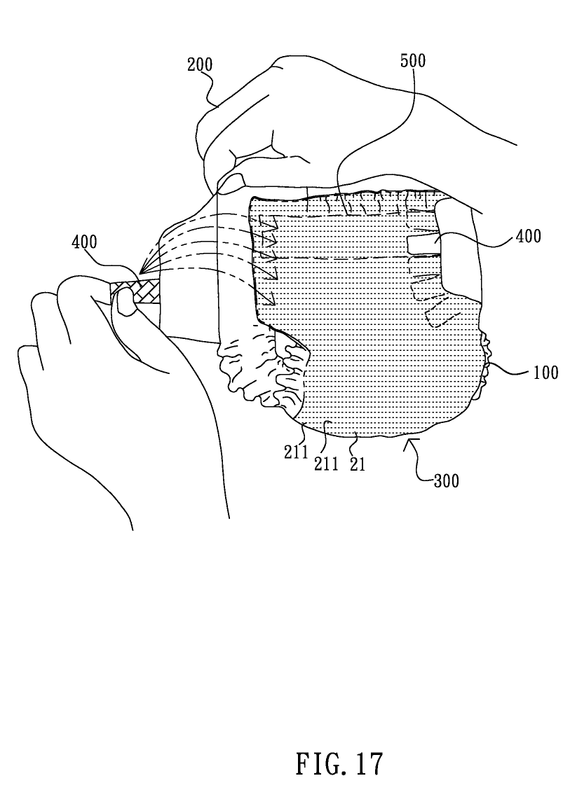

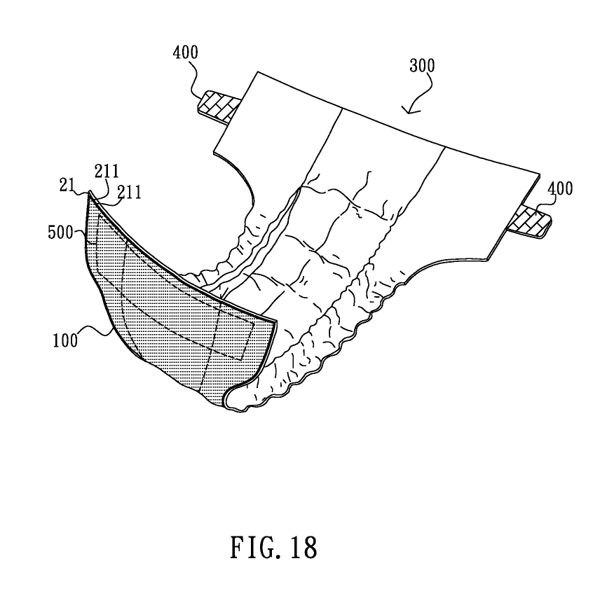

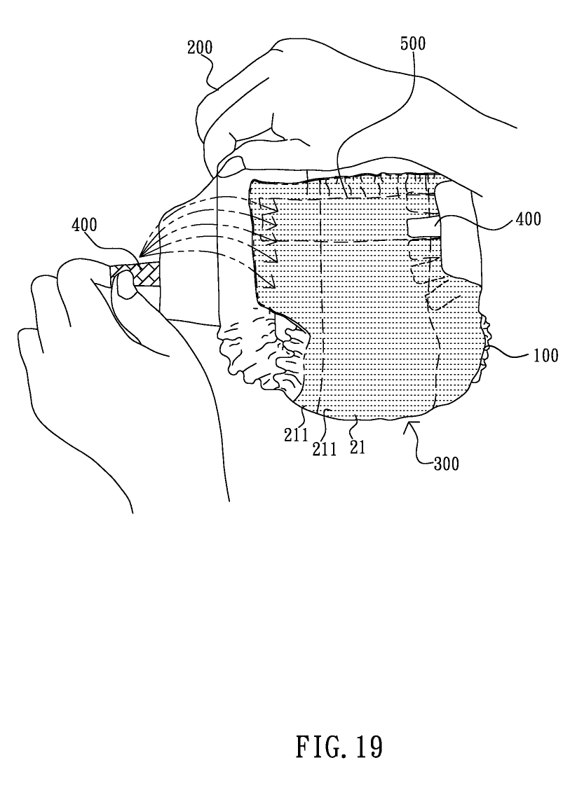

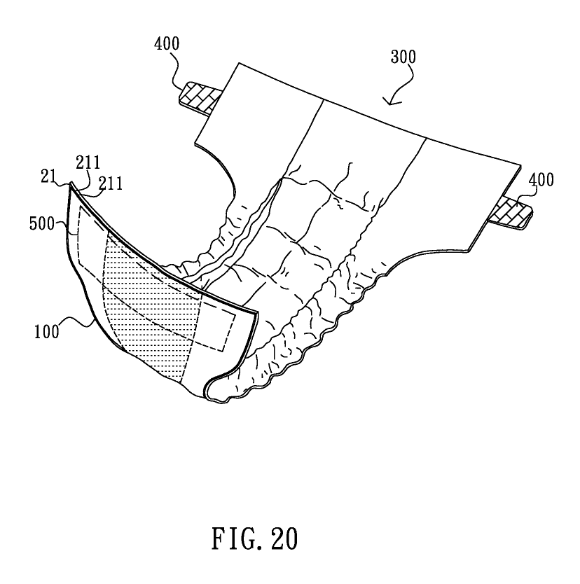

FIGS. 8, 10, 12 and 14 are front views of second, third, fourth and fifth models of the leakage proof base material for paper diaper of the present invention respectively; FIGS. 9, 11, 13 and 15 are cross-sectional partial enlarged views of FIGS. 8, 10, 12 and 14 respectively.

As shown in the drawings, the through hole areas 21 are disposed on the non-textile fabric layer 2 in one of the ways of: equally spaced at intervals, unequally spaced at intervals, covering the surface entirely, covering the surface partially, covering the central surface of the non-textile fabric layer 2 entirely, covering the central surface of the non-textile fabric layer 2 partially, covering the central surface of the non-textile fabric layer 2 in equally spaced intervals and covering the central surface of the non-textile fabric layer 2 in unequally spaced intervals.

By changing the position of the through hole area 21, many different choices are provided when the leakage proof base material of the present invention is applied in different types of paper diapers such as adult paper diaper and baby paper diaper.

The through hole area 21 is disposed in one of the shapes of: circular area, character area, strip area, rectangular area, pattern area, dot area, tartan area, regular striation area and irregular striation area.

With different shapes of the through hole area 21, the look can be aesthetically pleasing and the leakage proof base material 100 of the present invention can have a wider range of applications to be applied in different types of paper diaper.

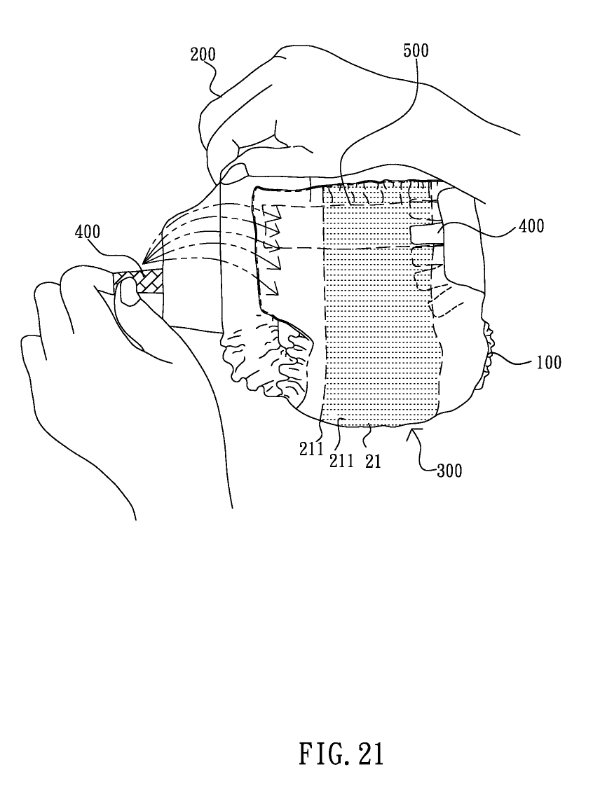

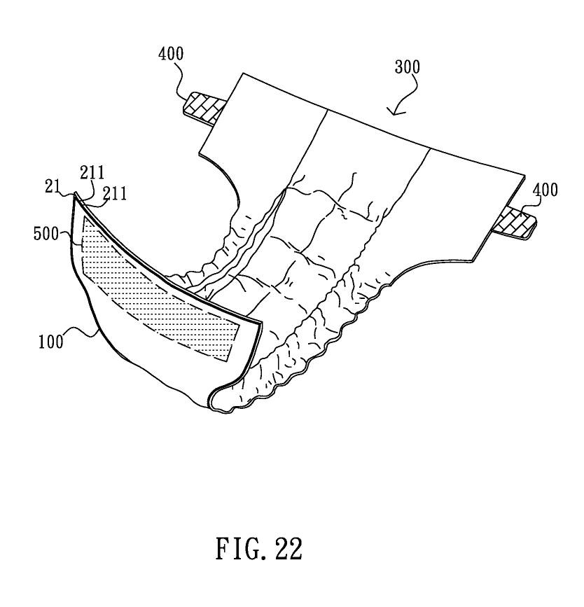

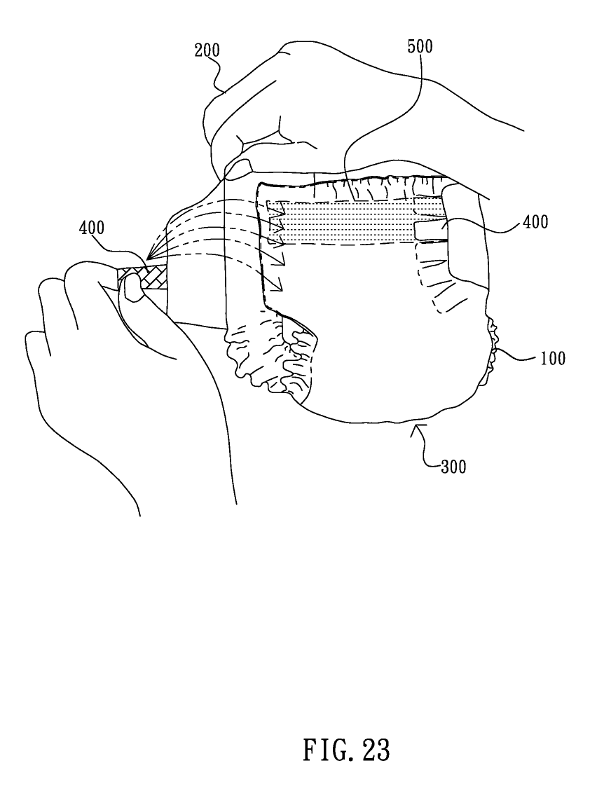

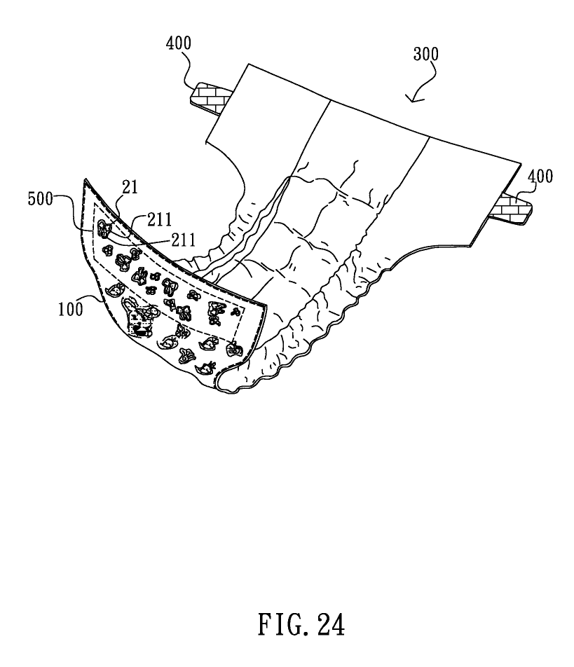

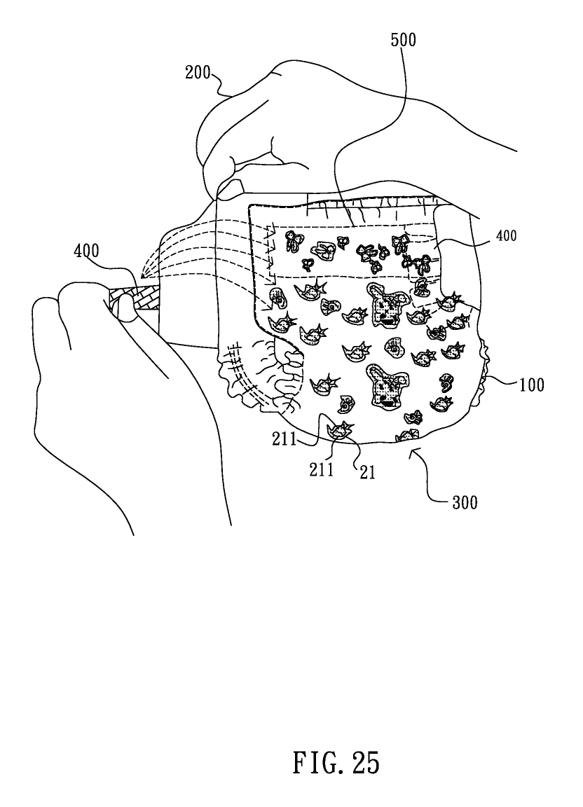

FIGS. 16 and 17 are perspective views of the first model of the leakage proof base material for paper diaper of the present invention being embodied on a paper diaper; FIGS. 18 and 19 are perspective views of the second model of the leakage proof base material for paper diaper of the present invention being embodied on a paper diaper; FIGS. 20 and 21 are perspective views of the third model of the leakage proof base material for paper diaper of the present invention being embodied on a paper diaper; FIGS. 22 and 23 are perspective views of the fourth model of the leakage proof base material for paper diaper of the present invention being embodied on a paper diaper; and FIGS. 24 and 25 are perspective views of the fifth model of the leakage proof base material for paper diaper of the present invention being embodied on a paper diaper.

As shown in the drawings of the first to the fifth models of the present invention, when the leakage proof base material 100 of the present invention is applied on a paper diaper 300, besides that the overall costs and the amount of materials used can be reduced, the skin of the baby or adult will not be adhered by the leakage proof base material 100, and the leakage proof base material 100 can be adhered repeatedly for convenience of checking. Even if skin moisturizing oil or water is spilled on the leakage proof base material 100, the leakage proof base material 100 and hooks 400 can be adhered together for fixing the paper diaper 300 on the skin.

Furthermore, by employing the through hole area 21 of the leakage proof base material 100, the discomfort of the skin can be eliminated when it is in contact with the leakage proof base material 100. The comfort provided by the present invention is like that of the cloth diaper made by textile fabric or cotton and therefore the skin-friendliness can be enhanced.

Comparing with the conventional paper diaper, the combination of the leakage proof base material 100 and the hooks 400 is very convenient already. In order that the present invention can be used in the way the conventional paper diaper being used by the users and for the sake of convenience of the users, besides the surface of the present invention can be added with colored patterns and trademarks, a target pattern area 500 can be printed on the leakage proof base material 100 to replace the original loop area.

However, the printing of the aforementioned patterns can be omitted based on the overall costs and requirements, and the target pattern area 50 can still be marked by using the through hole area 21.

With the target pattern area 50, besides that the conventional habit of the users can be met, the paper diaper being employed with the leakage proof base material 100 can be avoided to be misjudged by consumers as a defective product because the loop area is not disposed.

As a conclusion from the abovementioned, comparing the conventional paper diaper and the paper diaper with an improved combining structure of Taiwan patent publication number M405257 with the combination of the leakage proof membrane 1 and the non-textile fabric layer 2 and with the application of the through hole area 21, the cost and the defective rate of the present invention are lower and the amount of materials used can be reduced. Furthermore, the present invention is more convenient to use for the consumers, and has better applicability, practicability and functionality for the manufacturers.

Note that the specifications relating to the above embodiments should be construed as exemplary rather than as limitative of the present invention, with many variations and modifications being readily attainable by a person of average skill in the art without departing from the spirit or scope thereof as defined by the appended claims and their legal equivalents.

When introducing elements of the present invention or the embodiment(s) thereof, the articles “a,” “an,” “the,” and “said” are intended to mean that there are one or more of the elements. The terms “comprising,” “including,” and “having” are intended to be inclusive and mean that there may be additional elements other than the listed elements.

In view of the above, it will be seen that the several benefits of the invention are achieved and other advantageous results attained.

As various changes could be made in the above system and method without departing from the scope of the invention, it is intended that all matter contained in the above description and shown in the accompanying drawings shall be interpreted as illustrative and not in a limiting sense.

What is claimed is:

1. A leakage proof base material for paper diaper comprising:
a leakage proof base material composed of a leakage proof membrane as a top layer and a non-textile fabric layer as a bottom layer, said leakage proof membrane having surfaces combining together with surfaces of said non-textile fabric layer via one of the following methods: partially and entirely; and
at least one through hole area being disposed on the non-textile fabric layer, said through hole area comprising a plurality of through holes, said plurality of through holes being penetrable and being spaced with intervals.

2. The leakage proof base material as claimed in claim 1 wherein:
the leakage proof membrane and the non-textile fabric layer comprises one or more of the following materials: an elastic layer, a non-elastic layer, and a combination of an elastic layer and a non-elastic layer.

3. The leakage proof base material as claimed in claim 1, wherein the leakage proof membrane comprises one of the following: an air permeable leakage proof membrane and an air impermeable leakage proof membrane based on the degree of air permeability.

4. The leakage proof base material as claimed in claim 1, wherein the leakage proof membrane comprises one of the following: a polypropylene leakage proof membrane, a polyethylene leakage proof membrane, a polyurethane leakage proof membrane, and a plastic composite leakage proof membrane of decomposable plastic and copolymer.

5. The leakage proof base material as claimed in claim 1, wherein the through hole areas are disposed on the non-textile fabric layer in one of the ways of equally spaced at intervals, unequally spaced at intervals, covering the surface entirely, covering the surface partially, covering the central surface of the non-textile fabric layer entirely, covering the central surface of the non-textile fabric layer partially, covering the central surface of the non-textile fabric layer in equally spaced intervals and covering the central surface of the non-textile fabric layer in unequally spaced intervals.

6. The leakage proof base material as claimed in claim 1, wherein the through hole area is disposed in one of the shapes of the following: circular area, character area, strip area, rectangular area, pattern area, dot area, tartan area, regular striation area and irregular striation area.

7. The leakage proof base material as claimed in claim 1, wherein elasticity coefficients of the leakage proof membrane and the non-textile fabric layer being different.
